(19) United States
(12) Patent Application Publication
(10) Pub. No.: US 2009/0214587 A1
(43) Pub. Date: Aug. 27, 2009

(54) Recombinant virus and use thereof

(75) Inventors: Michinori Kohara, Tokyo (JP); Kyosuke Mizuno, Kumamoto (JP); Hisatoshi Shida, Hokkaido (JP); Kouji Matsushima, Tokyo (JP); Koichi Morita, Nagasaki (JP); Minoru Kidokoro, Tokyo (JP); Yukie Sameshima, Tokyo (JP)

Correspondence Address: DARBY & DARBY P.C., P.O. BOX 770, Church Street Station, New York, NY, 10008-0770, US

(73) Assignees: Post Genome Institute Co., Ltd., Tokyo (JP); The Chemo-Sero-Therapeutic Research Institute, Kumamoto-shi (JP)

(21) Appl. No.: 11/576,829

(22) PCT Filed: Oct. 11, 2005
(86) PCT No.: PCT/JP05/19151

§ 371 Date: Sep. 18, 2007

(30) Foreign Application Priority Data
Oct. 8, 2004 (JP) 2004-296734

(51) Int. Cl. A61K 39/215; C12N 7/00

(52) U.S. Cl. 424/199.1; 435/235.1

(57) ABSTRACT

The present invention provides a recombinant virus which is efficacious and highly safe in preventing the onset of SARS infection and a vaccine for SARS coronavirus containing the same. The recombinant virus of the invention can express a SARS coronavirus gene.

### CROSS-REFERENCE TO PRIOR APPLICATION

This is a U.S. National Phase Application under 35 U.S.C. §371 of International Patent Application No. PCT/JP2005/019151 filed Oct. 11, 2005, and claims the benefit of Japanese Patent Application No. 2004-296734, filed Oct. 8, 2004, both of which are incorporated by reference herein. The International Application was published in Japanese on Apr. 13, 2006 as WO 2006/038742 A1 under PCT Article 21(2).

### TECHNICAL FIELD

The present invention relates to a recombinant virus capable of expressing SARS coronavirus gene and use of this virus. More particularly, the present invention relates to a recombinant virus found by research and development of preventive medicine for SARS infection that became one of the most important and pressing problems in today's health service, and a vaccine for SARS coronavirus using the same.

### BACKGROUND ART

The global epidemic of SARS (atypical pneumonia, Severe Acute Respiratory Syndrome) coronavirus (SARS-CoV) that broke out in Guangzhou city, Guangdong province in China in February, 2003 gave evidence of threat posed by emerging virus infection in large cities, and the importance of general preventive measures against such infection. It would be fair to say that the worldwide development of transportation system covering and linking the large cities can accelerate such a disaster any moment.

The process from infection to onset of SARS-CoV has not yet been understood. Although the only measure taken today is isolation of infected individuals, it has been found that a serum of an infected individual can prevent the onset, showing effectiveness of vaccine against infection and onset of SARS-CoV (see, for example, Li Y, Xu J, Mo H Y, et al., Zhongguo Wei Zhong Bing Ji Jiu Yi Xue., 2004, vol. 16, p. 409-412). Thus, prompt development and production of a safer and highly effective vaccine against SARS infection and onset, the most important and urgent problem in today's health service, have been strongly desired.

Among various vaccines, a live vaccine is one of the particularly effective kinds. In general, development of an attenuated vaccine for an emerging virus is known to take a long time, and the same can be said for SARS-CoV. In such a case, a well-known genetic engineering procedure is employed in which a “recombinant vaccinia virus” is produced as a live vaccine. For example, recombinant vaccinia viruses for rabies virus and rinderpest developed by the present inventors are known (see, e.g., Tsukiyama K, Yoshikawa Y, Kamata H et al., Arch. Virol., 1989, vol. 107, p. 225-235), which have already been proved in a field testing to be excellently effective in preventing the infection and the onset.

A recombinant parent (vaccinia virus) used for producing a recombinant vaccinia virus needs to be a safety-confirmed vaccine strain. As such a vaccine strain, vaccinia virus strain LC16 m8 is known (see, e.g., Sugimoto M, Yasuda A, Miki K et al., Microbiol Immunol., 1985, vol. 29, p. 421-428). Strain LC16 m8 is derived from a Lister strain, and has been actually used for administration as a preventive vaccine, confirmed of its safety and effectiveness, and thus is the only vaccine strain generally produced at present.

Furthermore, during the research and development of the recombinant vaccinia viruses for rinderpest and HIV, the present inventors succeeded in developing a gene expression promoter capable of greatly enhancing the antibody-producing capacity and the cellular immunity-inducing capacity. Specifically, examples include plasmid vectors pSFJ1-10 and pSFJ2-16 (see, e.g., Jin N-Y, Funahashi S and Shida H, Arch. Virol., 1994, vol. 138, p. 315-330).

### DISCLOSURE OF INVENTION

The problem to be solved by the present invention is to provide a recombinant virus effective and highly safe in preventing SARS infection and onset, and a vaccine for SARS coronavirus comprising the same.

In order to solve this problem, the present inventors have gone through keen examination based on experience from years of research on infection of poliovirus, rinderpest virus, hepatitis C virus and the like, and making use of the achievement from the success in the development of a live vaccine for rinderpest. As a result, they succeeded in expressing SARS coronavirus protein from a vaccinia virus genome, thereby accomplishing the present invention.

Thus, the present invention is as follows.

(1) A recombinant virus capable of expressing SARS coronavirus gene.

In the recombinant virus of (1) above, the SARS coronavirus gene may comprise at least a structural protein gene of the virus, or the structural protein gene may comprise at least a spike protein gene of the virus. Moreover, the recombinant virus of (1) above may be, for example, capable of producing pseudo-SARS coronavirion or may be a vaccinia virus transformant, or the vaccinia virus may be strain LC16m8. In the recombinant virus of (1) above, the SARS coronavirus gene, for example, may be inserted into the HA gene region of the vaccinia virus genome, or may be inserted into the vaccinia virus genome so as to be placed downstream from a hybrid promoter.

(2) A vaccine for SARS coronavirus comprising the recombinant virus of (1) above.

### BRIEF DESCRIPTION OF DRAWINGS

FIG. 1 is a schematic view of plasmid vector “pSFJ1-10-SARS-S” obtained in Example 1 (structural view of SARS-CoV S protein expression vector).

ATI: A-Type Inclusion Body

MCS: Multi Cloning Site

vvHA: Vaccinia Virus Hemagglutinin Gene

TRS: Transcription Regulating Sequence

FIG. 2 is a picture of an agarose gel electrophoresis showing the results of confirmation of S protein gene transfer by PCR.

FIG. 3A is a schematic view showing the binding position between S probe (Spike probe) and HA probe used for plaque hybridization.

FIG. 3B is a picture of culture plates showing the results of confirmation of S protein gene transfer by plaque hybridization. As a result, only Sample 1 was positive.

FIG. 4 is a picture of PVDF membranes showing the results of confirmation of S protein expression (24 hours following infection) by western blotting.

FIG. 5 is a graph showing the results of assessment of neutralizing capacity against SARS coronavirus. X-axis represents the period from the virus inoculation, while Y-axis represents NT₅₀ measurements.

### DETAILED DESCRIPTION

Hereinafter, a recombinant virus and use thereof according to the present invention will be described in more detail. The scope of present invention, however, is not limited to the description, and the following illustrations may be modified and carried out without departing from the scope of the invention. The documents, the laid-open patent applications, the patent publications and other patent documents cited herein are incorporated herein by reference.

1. Recombinant Virus

A recombinant virus of the present invention is capable of expressing SARS coronavirus gene.

The recombinant virus of the present invention is preferably, but not limited to, what is called a recombinant vaccinia virus (a transformant of vaccinia virus parent) in which SARS coronavirus gene is integrated into the genome of the recombinant vaccinia virus parent such that it can express the protein. In general, a vaccinia virus refers to an “attenuated strain” which can be proliferated in an animal individual (animal cell) but with a very low proliferation in neuron.

Examples of the vaccinia virus as the recombinant parent include but not limited to vaccinia virus strain LC16 m8, Wyeth strain and Lister strain, among which vaccinia virus strain LC16m8 with very low proliferation in neuron is preferable. This is because strain LC16m8 is a safe and effective vaccine strain, which has been approved in Japan as smallpox vaccine, which has caused no serious side effect from vaccination of about 100,000 children (Research report from the Vaccination Research Group, Japanese Ministry of Health, Labour and Welfare, Clinical Virology, vol. 3, No. 3, p 269 (1975)) and whose immune-inducing capacity has been reported to be equal to that of the parent Lister strain (Morita M, Suzuki K, Yasuda A, et al., Vaccine, 1987, vol. 5, p. 65-70). Thus, the recombinant vaccinia virus of the present invention is preferably a transformant of strain LC16m8.

SARS coronavirus is an RNA virus having a 29751-base ssRNA genome, and the total DNA nucleotide sequence corresponding to this ssRNA is indicated in “GenBank No. NC_{—}004718”. Sequence regions coding for various proteins of SARS coronavirus are also indicated.

The SARS coronavirus gene integrated in the genome of the recombinant virus of the present invention is DNA gene. This DNA gene may be a DNA fragment obtained, for example, as follows: the ssRNA is isolated to obtain full-length cDNA using reverse transcriptase, which is used as a template to amplify and collect a protein gene region of interest by PCR. The nucleotide sequence of the DNA fragment may include a nucleotide sequence that is recognized as a transcription termination signal (e.g., “TTTTTNT”) in the recombinant vaccinia virus parent, and when this sequence is integrated directly into this virus genome, expression of the protein is decreased. In this case, mutation (silent mutation) is preferably introduced into part of the nucleotide sequence as the transcription termination signal before integration into the genome. Introduction of the mutation may be carried out by employing known site-directed mutagenesis (e.g., Quick-change kit (Stratagene, Model number: 200523)).

Specifically, SARS coronavirus gene that can be expressed in the recombinant virus of the present invention is either a structural protein gene or a nonstructural protein gene of the virus, but it preferably includes at least a structural protein gene. According to the present invention, the structural protein gene is a gene that defines the amino acid sequence of the structural protein after transcription and translation, and the structural protein refers to a protein whose functions are to form and maintain the structure and morphology in an organism. The nonstructural protein gene is a gene that defines the amino acid sequence of the nonstructural protein after transcription and translation, and the nonstructural protein refers to a protein capable of exerting functions other than the functions of the structural gene exerted in an organism.

Examples of the structural protein gene of SARS coronavirus include spike protein (S protein) gene, membrane protein (M protein) gene, envelope protein (E protein) gene and nucleocapsid protein (N protein) gene, among which those that include S protein gene (i.e., those that require S protein gene) are preferable. This is because when S protein expressed from at least S protein gene is used as an antigen, a recombinant virus can serve as a virus with a good immune-inducing property.

Structural protein gene containing only the S protein gene and structural protein gene containing a combination of the S protein gene and other structural protein gene are equally preferable. In the latter case, the other structural protein gene is preferably, for example, N protein gene, M protein gene and E protein gene, and one of the preferable embodiments is a combination capable of producing pseudo-SARS coronavirion as described below.

Examples of the nonstructural protein gene of SARS coronavirus include helicase and protease.

Preferably, but without limitation, the recombinant virus of the present invention can produce pseudo-SARS coronavirion. Use of this pseudovirion as an antigen is expected to provide a recombinant virus with exceptional immune inducing property.

Specifically, a pseudo-SARS coronavirion is not a virion with virulence characteristic of SARS coronavirus, but, for example, is one that does not have the virulence but is similar to this virion in structural point of view.

If the recombinant virus of the present invention is to produce a pseudo-SARS coronavirion, SARS coronavirus gene should include at least N protein gene, M protein gene and E protein gene of its structural protein gene so that these genes are integrated into the virus genome to be in a state capable of expressing the proteins corresponding to these genes.

The recombinant vaccinia virus of the present invention may be produced, but without limitation, by employing a conventional homologous recombinant technique.

For example, a plasmid vector containing “DNA sequence in which a foreign gene (SARS coronavirus gene) and a promoter for expressing the same are inserted into a sequence of a vaccinia virus genome, i.e., a recombinant parent (preferably, a sequence of a gene that is not necessary for proliferation of this virus (referred to as Gene a))” is constructed. The homologous recombinant of this plasmid vector and the vaccinia virus parent results in production of a recombinant vaccinia virus in which the promoter and the SARS coronavirus gene are inserted into the genome of the vaccinia virus parent (preferably, into the sequence of Gene a). The homologous recombination can be performed according to a conventional transfection technique. After infecting cultured animal cell (e.g., monkey kidney cell CV-1 or rabbit kidney cellRK13) with the vaccinia virus parent, the plasmid vector is transfected into the infected cell by calcium phosphate method or the like to obtain candidate recombinant viruses. Then, from these candidate recombinant viruses, the recombinant vaccinia virus of interest is obtained through various selection methods and confirmation tests.

Specifically, for example, “hemagglutinin (HA) gene” is preferably selected as Gene a in the genome of the vaccinia virus parent such as strain LC16m8. For example, in a genome of strain LC16m8 or the like, generally, thymidine kinase (TK) gene region is often used as an insertion site for a foreign gene among regions other than the HA gene region. When a foreign gene or the like is inserted into the TK gene region, defective expression of TK protein reduces proliferation of the recombinant vaccinia virus. On the other hand, defective expression of HA protein has little influence on the proliferation, and thus benefits inherent in strain LC16m8 or the like as the vaccinia virus parent can be fully exerted. In addition, when the HA gene region in the genome of strain LC16m8 or the like is inserted with a foreign gene or the like by homologous recombination, HA protein is not expressed and thus hemagglutination characteristic of HA protein does not occur. A recombinant vaccinia virus of interest obtained by homologous recombination can readily and efficiently be screened as follows: animal monolayer cells (e.g., RK13 cells) are infected with the candidate recombinant virus following transfection to form plaques, to which an erythrocyte (e.g., chicken erythrocyte) solution is added, and then a plaque without hemagglutination (i.e., white plaque (HA⁻)) is sorted and collected.

A plasmid vector used for the homologous recombination is not limited as long as it contains the DNA sequence having the properties described above. In general, according to a conventional gene recombination technique, a known plasmid vector is used as a parent, which is constructed by inserting a DNA sequence having the characteristics described above, or by appropriately inserting only DNA sequences necessary to acquire the same state as inserting the DNA having the characteristics. Examples of the plasmid vectors that act as the parent include pSFJ1-10 (Arch. Virol., 1994, vol. 138, p. 315-330, Japanese Laid-Open Application No. 6-237773 (Example 1-3)) and pSFJ2-16, among which pSFJ1-10 is preferable.

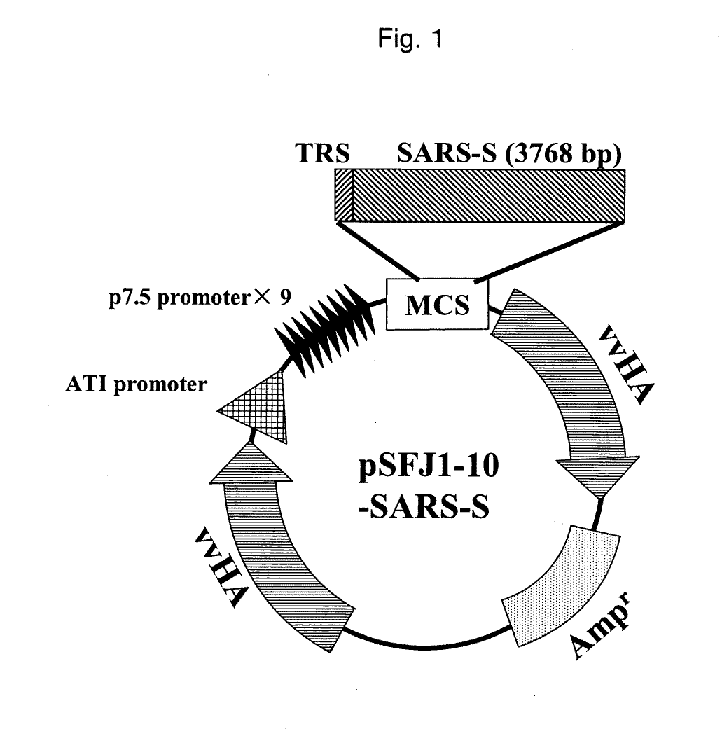

Plasmid vector pSFJ1-10 includes ‘DNA sequence having a multi cloning site and a hybrid promoter including a “poxvirus A-type inclusion body (ATI) promoter” and “repeats of expression mutation promoters of vaccinia virus strain LC16m8 7.5 kDa protein (p7.5)” in a hemagglutinin (HA) gene region of strain LC16m8 corresponding to Gene a’. When this plasmid vector is used, a recombinant vector can be constructed in which a SARS coronavirus gene of interest is inserted into the desired restriction enzyme site in the multi cloning site downstream from the hybrid promoter (see FIG. 1). Moreover, homologous recombination using this recombinant vector is performed to insert the SARS coronavirus gene into the genome (into the HA gene region) of the vaccinia virus parent so as to place it downstream from the hybrid promoter (specifically, together with the hybrid promoter). As a result, protein corresponding to the inserted SARS coronavirus gene can be expressed continuously and in a large quantity from early to later stage of recombinant vaccinia virus infection and with complete glycosylation modification.

2. Vaccine for SARS Coronavirus

A vaccine for SARS coronavirus according to the present invention includes the recombinant virus of the present invention. Since the recombinant virus of the present invention is safe, a vaccine for SARS coronavirus can be used not only as a preventive drug for preventing SARS infection in advance but also as a therapeutic agent for relieving the symptoms resulting from SARS infection.

In order to be used as a live vaccine, a vaccine for SARS coronavirus generally includes components other than the recombinant virus of the present invention. Examples of other components include: water; oil phase containing at least one type of oil (if possible, in an emulsified form); ester obtained by condensation of sugar or glycerol and fatty acid; and an emulsified form containing a derivative of the ester. Either only one component or two or more components can be used.

The content percentage of the recombinant virus of the present invention in a vaccine for SARS coronavirus, either as a SARS preventive drug or a SARS therapeutic agent, is generally but without limitation preferably 30% or higher, more preferably 50% or higher, and still more preferably 80% or higher. When the content percentage is within the above range, advantages can be obtained such as efficient enhancement of the immunity of the host and increase in the neutralizing antibody titer or cytotoxicity.

Generally, inoculation modes of the vaccine for SARS coronavirus, either as a SARS preventive drug or a SARS therapeutic agent, include but not limited to transdermal inoculation (preferably intradermal inoculation), intramuscular inoculation and transnasal inoculation, or may include, for example, oral inoculation.

Forms of the vaccine for SARS coronavirus, either as a SARS preventive drug or a SARS therapeutic agent, include but not limited to an injectable agent (a subcutaneous injectable agent, etc.), an intramuscular injectable agent, oral preparation, oral spray preparation and transnasal spray preparation, among which an injectable agent is preferable.

When used as an injectable agent, either as a SARS preventive drug or a SARS therapeutic agent, the dose of a vaccine for SARS coronavirus is preferably but without limitation, for example, 10²-10¹⁰ PFU/body.

When used as an oral preparation, either as a SARS preventive drug or a SARS therapeutic agent, the dose of a vaccine for SARS coronavirus is preferably but without limitation, for example, 10⁴-10¹² PFU/body.

Hereinafter, the present invention will be described more specifically by way of examples, although the present invention is not limited thereto. Hereinafter, for the sake of convenience, “% by weight” is also indicated as “wt %”.

### Example 1

### Preparation of Recombinant Vaccinia Virus (RVV-S)

First, S protein gene was isolated and prepared as follows. SARS coronavirus was proliferated in animal cell (Vero cell), and then full-length RNA (complete genome, 29751 base, ssRNA) was extracted and isolated according to a conventional technique to synthesize cDNA with reverse transcriptase. Then, primers represented by SEQ ID NOS:1 and 2 specific to S protein gene (sequence 21482-25259 in the entire nucleotide sequence of the SARS coronavirus indicated by “GenBank No. NC_{—}004718”) and the cDNA as a template were used for PCR. According to this PCR, composition of a reaction solution was 1 U DNA polymerase, 0.3 mM dNTP, 1 μM F primer and 1 μM R primer in 50 μL buffer accompanying commercially available polymerase while the cycle conditions were 25 cycles of melting at 95° C. for 0.5 minutes, annealing at 58° C. for 0.5 minute and elongation at 72° C. for 2 minutes.

F primer (SEQ ID NO:1):
5′-GGGCGGCGAA TTCCTAAACG AACATGTTTA TTTTCTTATT
ATTTCTTACT CTC-3′
R primer (SEQ ID NO:2):
5′-GGGCGGCGAA TTCTTATGTG TAATGTAATT TGACACCCTT
GAG-3′

Moreover, sequences “TTTTTNT” are present at two positions in the S protein gene. Since this sequence becomes a transcription termination signal for the promoter in a vaccinia virus (see Virol., 1991, vol. 185, p. 432-436), mutation (silent mutation) was introduced into bases of codons including part of the sequence “TTTTTNT” without altering the amino acid sequence of S protein. Specifically, Quick-change kit (Stratagene) was used to change the nucleotide sequence “TTTTTTT” at 22569-22575 into “TTCTTCT” and the nucleotide sequence “TTTTTGT” at 25580-25586 into “TCTTCGT”.

Then, plasmid vector pSFJ1-10 (Arch. Virol., 1994, vol. 138, p. 315-330, Japanese Laid-Open Application No. 6-237773 (Examples 1-3)) was prepared. Plasmid vector pSFJ1-10 has a multi cloning site and a hybrid promoter including a “poxvirus A-type inclusion body (ATI) promoter” and “repeats of expression mutation promoters of vaccinia virus strain LC16m8 7.5 kDa protein (p7.5)” in a hemagglutinin (HA) gene region of vaccinia virus strain LC16m8. This hybrid promoter expresses a protein in a large quantity from early to later stage of recombinant vaccinia virus infection and with complete glycosylation modification.

Following a conventional genetic recombinant technique, S protein gene of SARS coronavirus was integrated into KpnI site of the multi cloning site of pSFJ1-10 to insert S protein gene into the hemagglutinin (HA) gene region of pSFJ1-10 downstream from the ATI/p7.5 hybrid promoter, thereby preparing novel plasmid vector pSFJ1-10-SARS-S (see FIG. 1).

Then, RK13 cell (in this example, “primary-cultured kidney cell” may be used instead of this and the following “RK13 cells”) was seeded to a T175 flask. Once it had grown to a confluent state, it was infected with vaccinia virus strain LC16m8 at moi=10 at 30° C. for 2 hours. Following infection, the virus solution was suctioned away, and the cells were washed with PBS(−).

Thereafter, 10 mL of 0.05% trypsin/0.5 mM EDTA/PBS(−) solution was added to the cells, which were incubated at 37° C. for 1 minute, washed with 10% FCS/MEM medium, PBS(−), HeBS buffer, and suspended in 600 μL HeBS buffer to obtain a cell suspension.

Forty μg of plasmid vector pSFJ1-10-SARS-S was diluted with HeBS buffer to a total amount of 200 μL, which was added to and mixed with the cell suspension, and left to stand on ice for 10 minutes. This cell suspension was transferred to a 0.4 cm cuvette and subjected to electroporation (0.2 kV, 960 F) using an electroporator (Bio-Rad, Product name: Gene-Pulser).

Following electroporation, 1 mL of 10% FCS/MEM medium was immediately added to the cell suspension, which was added to a T175 flask that had been seeded with RK13 cells beforehand and cultured at 30° C. for 24 hours.

Following cultivation, the cells were scraped with a scraper to obtain a cell suspension. This cell suspension was collected, and subjected to ultrasonication (30 seconds×4 times) in cold water (about 4° C.) followed by centrifugation (2000 rpm, 10 minutes). The supernatant obtained after the centrifugation was used as a virus solution. This virus solution was diluted with 1.9 mL 10% FCS/MEM medium, added to a 100 mm dish that had been seeded with RK13 cells beforehand for infection at 30° C. for an hour. Then, the virus solution was suctioned away, the cells were washed with PBS(−), added with 10 mL 10% FCS/0.5% methylcellulose/MEM medium and cultured at 30° C. for 72 hours.

Following cultivation, the supernatant was suctioned away and the cells were washed with PBS(−). Ten mL of chicken erythrocyte solution diluted in PBS(+) (concentration: 0.5%) was added to the 100 mm dish and cultured at 37° C. for 30 minutes. Then, the erythrocyte solution was suctioned away and the cells were washed twice with PBS(−).

Following washing, plaques that were not adsorbed with chicken erythrocyte were collected using a Pipetman. Specifically, when the HA gene region of vaccinia virus strain LC16m8 was inserted with S protein gene by homologous recombinantion involving pSFJ1-10-SARS-S, HA protein was not expressed and thus hemagglutination inherent in HA protein did not occur. Thus, after addition of the chicken erythrocyte solution, plaques showing no hemagglutination, i.e., white plaque, can be selected to efficiently collect recombinant vaccinia virus of interest introduced with the S protein gene.

For the virus in the collected plaque, introduction of S protein gene was confirmed by PCR and plaque hybridization in the same manner as the method described below. For virus confirmed with gene transfer, plaque purification described above was repeated for three times.

Plaques obtained after the third purification was suspended in 700 μL 10% FCS/MEM medium, and subjected to ultrasonication (30 seconds×4 times) in cold water. Centrifugation (2000 rpm, 10 minutes) was performed and 500 μL supernatant was added to a T25 flask that had been seeded with RK13 cells beforehand for infection at 30° C. for 2 hours. After the infection, virus solution was suctioned away, and the cells were washed with 2.5 mL 10% FCS/MEM medium. After suctioning the medium away, 2.5 mL 10% FCS/MEM medium was further added and cultured at 30° C. for 72 hours.

After the cultivation, the cells were scraped using a scraper to collect the cell suspension. This cell suspension was subjected to ultrasonication (30 seconds×4 times) in cold water, followed by centrifugation. The supernatant was collected as a virus solution.

The collected virus solution was subjected to serial dilution, and added to a 6 well plate that had been seeded with RK13 cell beforehand for infection at 30° C. for an hour. Then, the virus solution was suctioned away, the cells were washed twice with PBS(−), added with 2 mL 10% FCS/0.5% methylcellulose/MEM medium and cultured at 30° C. for 72 hours.

After the cultivation, the number of plaques formed in the well was counted to calculate the titer. The titer of the original solution (PFU/mL) can be obtained by multiplying the number of plaques in the well by the dilution rate to calculate PFU contained in 1 mL of the original solution.

Based on the calculated titer, moi was adjusted considering the number of PFU and the number of cells in the T175 flask, to perform large scale cultivation as described below.

RK13 cells were seeded to ten T175 flasks. Once the cells had grown to a confluent state, they were infected with the recombinant vaccinia virus solution at moi (multiplicity of infection (PFU per cell)) of 0.1 at 30° C. for 2 hours. After the infection, the virus solution was suctioned away, and the cells were washed with 20 mL 10% FCS/MEM medium. The medium was suctioned away, and 20 mL 10% FCS/MEM medium was further added to culture at 30° C. for 72 hours.

After the cultivation, the cells were scraped from the flask using a scraper to collect and freeze the cell suspension at −80° C. for preservation. After repeating the freezing and thawing for three times, the cell suspension was subjected to ultrasonication (30 seconds×4 times) in cold water, followed by centrifugation (2000 rpm, 10 minutes) to collect the supernatant as a virus solution.

The collected virus solution was loaded into a high-speed centrifuge tube to perform centrifugation (18000 rpm, 45 minutes) to precipitate the virus. After suctioning the supernatant away, the pellets (viruses) were again suspended in a small amount of MEM medium. By doing so, a virus solution was prepared that was ten times stronger than the one obtained by cultivation in the T175 flask.

This concentrated virus solution was serial diluted and the titer thereof was calculated in the same manner as described above.

The following confirmatory experiment and evaluation were conducted for the recombinant vaccinia virus whose titer was determined.

<Confirmation of Transfer of S Protein Gene by PCR>

Primers represented by SEQ ID NOS:3 and 4 specific to S protein gene and the obtained recombinant vaccinia virus genome as a template were used in PCR to confirm the transfer of S protein gene in the virus genome.

F primer (SEQ ID NO:3): 5′-GGCTATGGCT GTCTTTCCTG-
3′
R primer (SEQ ID NO:4): 5′-CAAGCGAAAA GGCATCAGAT
ATG-3′

Specifically, composition of the reaction solution was 1 U DNA polymerase, 0.3 mM dNTP, 1 μM F primer and 1 μM R primer in 50 μL buffer accompanying commercially available polymerase while the cycle conditions were 25 cycles of melting at 95° C. for 0.5 minutes, annealing at 58° C. for 0.5 minutes and elongation at 72° C. for 2 minutes. The resulting PCR product was subjected to electrophoresis using agarose gel to confirm the bands. Accordingly, if a single band at about 300 bp that was expected upon primer designing can be observed, S protein gene is introduced in the recombinant virus genome while S protein gene is not introduced if this band cannot be observed.

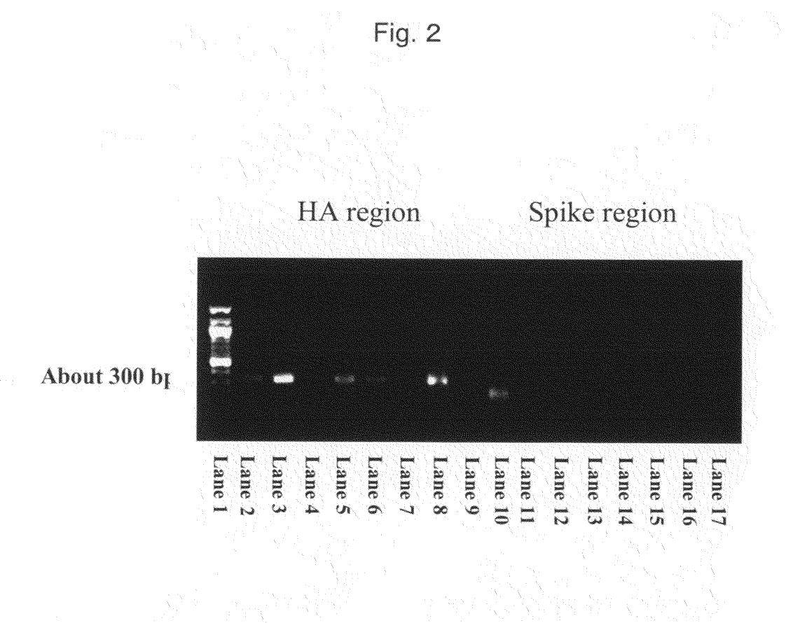

As can be appreciated from FIG. 2, the PCR product (amplified fragment) was subjected to electrophoresis with 2 wt % agarose gel, as a result of which, S protein gene was found to be introduced in the recombinant virus genome of the sample in Lane 10.

<Confirmation of Introduction of S Protein Gene by Plaque Hybridization>

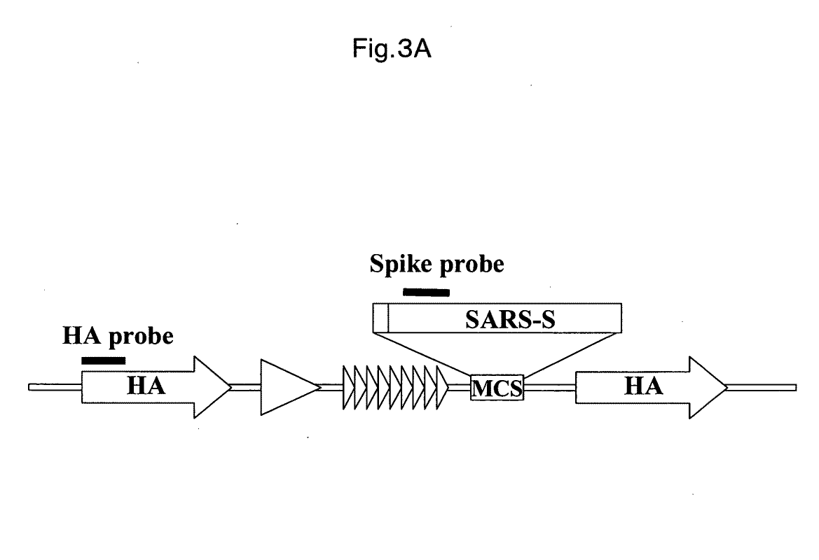

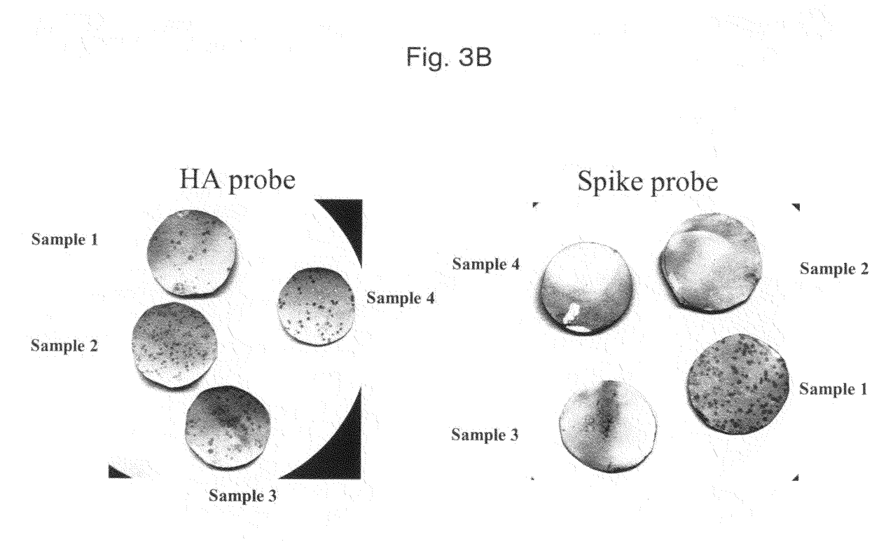

For plaques formed by infecting animal cell, a probe (Dig-dUTP-labeled) represented by SEQ ID NO:5 specific to S protein gene and a probe (Dig-dUTP-labeled) represented by SEQ ID NO:6 specific to HA gene were used for plaque hybridization technique to confirm whether S protein gene had been introduced into the genome of the resulting recombinant vaccinia virus (see FIG. 3A).

S probe (SEQ ID NO:5):
GACTTCTAACGCCATCGATGTTTAGATCCATCACACAAATACGAT
HA probe (SEQ ID NO:6):
GGTTCTACCATGAACAACAAGTCACAGTCGGTGATTATTATTAAC

Specifically, a recombinant virus solution was added to 6 well plates that had been seeded with RK13 cells beforehand for infection at 30° C. for an hour. Following infection, the virus solution was suctioned away, and the cells were washed with PBS(−) twice. To each well, 2 mL of 10% FCS/0.5% methylcellulose/MEM medium was added and cultured at 30° C. for 72 hours. After the cultivation, the medium was suctioned away and the cells were washed with PBS(−). The formed plaque was transferred onto a nylon membrane (Hybond N+) (Amersham), treated with a denaturing solution (0.5 M NaOH, 1.5 M NaCl) for 7 minutes, and then washed with a neutralizing solution (1.5 M NaCl, IM Tris-HCl) and 2×SSC solution. The nylon membrane was air-dried for 45 minutes, and subjected to UV crosslink using UV Stratalinker 2400 (Stratagene) by Auto crosslink. A rapid hybri buffer (Amersham) was added for 0.15 mL per cm² of the nylon membrane, heated at 65° C. for 30 minutes, and added with S probe or HA probe to 50 g/mL (final concentration). Hybridization was performed by heating at 95° C. for 10 minutes, and then lowering the temperature to 37° C. by 1° C. a minute. After washing with 2×SSC, 1×SSC and 0.1×SSC, 1% Blocking Reagent (Roche) was added for blocking for 30 minutes. Alkaline-phosphatase-labeled anti-Dig antibody was added to 0.2 μg/mL (final concentration) for treatment for 30 minutes followed by washing. NBT and X-phosphate were used for color development. Accordingly, if a color development in the shape of the plaque appears on the S-probe-treated nylon membrane, S protein gene is introduced in the recombinant virus genome while no color development means that S protein gene is not introduced. As can be appreciated from FIG. 3B, plaque hybridization indicates introduction of S protein gene in the recombinant virus genome of Sample 1.

<Confirmation of S Protein Expression by Western Blotting>

RK13 cells seeded on 6 well plates in advance were infected with the recombinant vaccinia virus at moi=30 at 30° C. for 2 hours. Following infection, the virus solution was suctioned away and the cells were washed twice with PBS(−). To each well, 2 mL of 10% FCS/MEM medium was added and cultured at 30° C. for 24 hours.

Following cultivation, the medium was suctioned away, and 100 μL lysis buffer (1% SDS, 0.5% NP-40, 0.15 M NaCl, 10 mM Tris-HCl (pH7.4)) was added to lyse the cells. This solution was transferred into a 1.5 mL Eppendorf tube. The collected solution was subjected to ultrasonication (30 seconds×4 times) in cold water until its viscosity disappeared. The amount of protein in the solution was quantified by Lowry method.

Using 7.5 wt % acrylamide gel, electrophoresis was performed using the solution as a sample. This electrophoresis was performed on 50 μg of protein for each lane.

At the end of the electrophoresis, the removed gel was applied with current of 5.5 mA/cm² for 60 minutes using a Semi-dry blotter (BIO-RAD, Model number: Trans-Blot (Registered Trademark) SD Cell), and protein in the gel was transferred onto a PVDF membrane. Then, the PVDF membrane was washed with TBS-T solution, immersed in 5 wt % skim milk-TBS-T solution for 180 minutes for blocking. At the end of the blocking, the PVDF membrane was washed for three times with TBS-T solution, added and reacted with a primary antibody solution.

IgG antibody was used as the primary antibody, which was obtained as follows: two types of peptides A and B represented by SEQ ID NOS: 7 and 8 were prepared based on the amino acid sequence of S protein; these peptides were used for immunization of a rabbit to prepare an antiserum (SIGMA GENOSYS, Product name: ST1168, ST1170) from which the IgG antibody was obtained by purification with Protein A using an Ampure PA kit (Amersham, Model number: RPN. 1752). The purified antibody was quantified by Lowry method, prepared and used in 10 μg/mL.

Peptide A (SEQ ID NO:7): CTDSVRDPKTSEI
Peptide B (SEQ ID NO:8): CKFDEDDSEPVLK

At the end of the reaction with the primary antibody, the PVDF membrane was washed for three times with TBS-T solution, and added and reacted with a secondary antibody solution.

As the secondary antibody, donkey anti-rabbit IgG-linked HRPO (Amersham, Product name: NA9340) was used.

At the end of the reaction with the secondary antibody, the PVDF membrane was again washed for three times with TBS-T solution. Thereafter, an ECL solution was added to the PVDF membrane, and the membrane was exposed for 3 minutes and a film was developed.

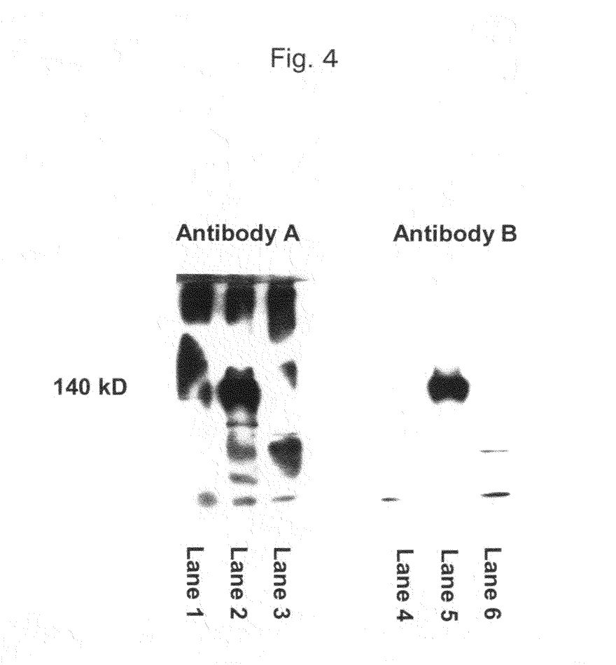

As a result, as shown in FIG. 4, samples in Lanes 2 and 5 were found to express S protein (140 kD) in the recombinant virus genome.

### Example 2

### Evaluation of Neutralizing Capacity Against SARS Coronavirus

<Virus Inoculation>

The recombinant vaccinia virus and the vaccinia virus strain LC16m8 obtained in Example 1 were separately inoculated transdermally to different rabbits (New Zealand white, female) at 1×10⁸ PFU and blood was taken from each ear vein after 1, 2, 3, 4 and 6 weeks.

Six weeks after the first virus inoculation, the same virus as the first inoculation was inoculated again at 1×10⁸ PFU. Similarly, at the end of the second inoculation, blood was taken from each ear vein after 1, 2, 3, 4 and 6 weeks.

All bloods were taken into evacuated blood collection tubes (TERUMO, Product name: Venoject II vacuum blood collection tube (sterilized), 9 mL), and centrifuged (3000 rpm, 20 minutes) to separate and collect the sera. The sera were frozen and stored at −20° C. until the neutralizing activity test against SARS coronavirus described below.

<Evaluation of Neutralizing Capacity>

The sera frozen and stored were melted at 37° C. and inactivated at 56° C. for 30 minutes to be used as serum samples for evaluating neutralizing capacity.

To SARS coronavirus of 200 TCID₅₀ obtained by subculturing Hanoi 01-03 strain in a non-serum medium (MEM) twice, the serum sample was added, left at 37° C. for an hour followed by 4° C. for an hour, and used for infecting Vero E6 cells that had been seeded onto a 96 well plate in advance.

Five days after the infection, CPE (cytopathic effect) was observed, the neutralization titer (NT₅₀) of each serum sample as an index of its neutralizing capacity were visually determined for evaluation.

Accordingly, NT₅₀ value of 10 or higher indicates that the anti-serum has neutralizing capacity.

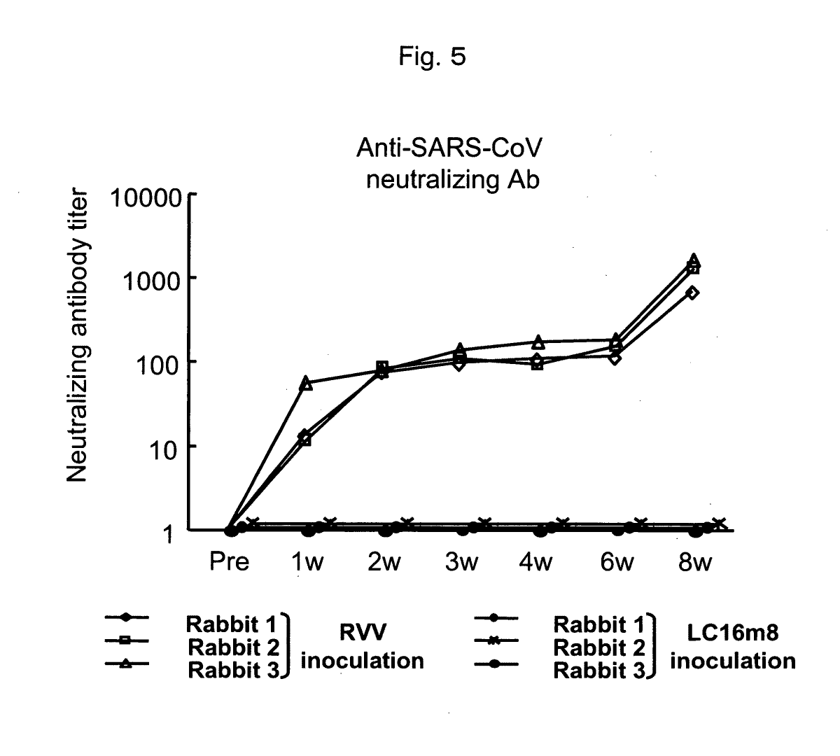

As shown in FIG. 5, in the serum of rabbit inoculated with the recombinant vaccinia virus obtained in Example 1, neutralizing antibody against SARS coronavirus were induced and produced at a very early stage of a week following inoculation, showing effective neutralizing activity (NT₅₀=26). Moreover, enhancement of the neutralizing activity was observed until 3 weeks following inoculation (NT₅₀=114). No vaccine has been reported to induce and produce neutralizing antibody against SARS coronavirus within a week following inoculation, and the neutralizing antibody titer induced by the recombinant vaccinia virus obtained in Example 1 is comparative to the strongest titer reported so far. When second inoculation was performed 6 weeks following first inoculation, the neutralizing activity of the serum obtained after 2 weeks was found to have increased by about 10 times (NT₅₀=1172) (compared to one obtained six weeks following first inoculation).

Accordingly, in an emergency of SARS resurgence, prompt action can be taken using live vaccines.

On the other hand, in a serum of a rabbit inoculated with strain LC16 m8, no neutralizing activity against SARS coronavirus was observed at any level after the inoculation.

Embodiments other than these examples include a vaccine that can express not only S protein of SARS coronavirus but also a plurality of structural proteins or nonstructural proteins at the same time. The present inventors have confirmed expression of S protein in a gene expression system that was constructed to express four types of structural proteins (S protein, M protein, E protein and N protein), in which case, pseudo-SARS coronavirion is also expected to be produced, showing the potential of a strong vaccine effect.

### INDUSTRIAL APPLICABILITY

The present invention provides a novel recombinant virus which is efficacious and highly safe in preventing the onset of SARS infection and a vaccine for SARS coronavirus containing the same.

Other Information:

SEQ ID NO: 1: primer

SEQ ID NO:2: primer

SEQ ID NO:3: primer

SEQ ID NO:4: primer

SEQ ID NO:5: probe

SEQ ID NO:6: probe

SEQ ID NO:7: peptide

SEQ ID NO:8: peptide

1. A recombinant virus capable of expressing Severe Acute Respiratory Syndrome (SARS) coronavirus gene.

2. The recombinant virus according to claim 1, wherein the gene comprises at least a structural protein gene of SARS coronavirus.

3. The recombinant virus according to claim 2, wherein the structural protein gene comprises at least a spike protein gene of SARS coronavirus.

4. The recombinant virus according to claim 1, capable of producing pseudo-SARS coronavirion.

5. The recombinant virus according to claim 1, comprising a transformant of vaccinia virus.

6. The recombinant virus according to claim 5, wherein the vaccinia virus is strain LC16m8.

7. The recombinant virus according to claim 5, wherein the SARS coronavirus gene is inserted into the HA gene region of the genome of the vaccinia virus.

8. The recombinant virus according to claim 5, wherein the SARS coronavirus gene is inserted into the genome of the vaccinia virus so as to be placed downstream from a hybrid promoter.

9. A vaccine for SARS coronavirus comprising the recombinant virus according to claim 1.


SEQUENCE LISTING (8 sequences)

SEQ ID NO: 1 - DNA, 53 bp, Artificial
  Primer
gggcggcgaattcctaaacgaacatgtttattttcttattatttcttactctc

SEQ ID NO: 2 - DNA, 43 bp, Artificial
  Primer
gggcggcgaattcttatgtgtaatgtaatttgacacccttgag

SEQ ID NO: 3 - DNA, 20 bp, Artificial
  Primer
ggctatggctgtctttcctg

SEQ ID NO: 4 - DNA, 23 bp, Artificial
  Primer
caagcgaaaaggcatcagatatg

SEQ ID NO: 5 - DNA, 45 bp, Artificial
  Probe
gacttctaacgccatcgatgtttagatccatcacacaaatacgat

SEQ ID NO: 6 - DNA, 45 bp, Artificial
  Probe
ggttctaccatgaacaacaagtcacagtcggtgattattattaac

SEQ ID NO: 7 - protein, 13 aa, Artificial
  Synthetic Peptide
CTDSVRDPKTSEI

SEQ ID NO: 8 - protein, 13 aa, Artificial
  Synthetic Peptide
CKFDEDDSEPVLK